(19) United States
(12) Patent Application Publication
(10) Pub. No.: US 2022/0313499 A1
(43) Pub. Date: Oct. 6, 2022

(54) Absorbent and Leak Proof Garments for Incontinence, Menstruation and Reproductive System Discharge

(71) Applicant: Clements Inceptum, LLC, Memphis, TN (US)

(72) Inventor: Cristian Clements, Memphis, TN (US)

(73) Assignee: Clements Inceptum, LLC, Memphis, TN (US)

(21) Appl. No.: 17/696,708

(22) Filed: Mar. 16, 2022

Related U.S. Application Data
(60) Provisional application No. 63/161,516, filed on Mar. 16, 2021.

(51) Int. Cl. A61F 13/496; D01F 6/70; A61F 13/49

(52) U.S. Cl. CPC A61F 13/496; D01F 6/70; A61F 13/49011; A41B 2400/22

(57) ABSTRACT

A garment for absorbance and leak retention of bodily fluids discharged from a body of a wearer. The garment has a main body and a protection panel lining an inner surface of the main body. The protection panel comprises a lower layer of thermoplastic polyurethane (TPU), an upper layer of a fluid permeable material, and an absorbent layer sandwiched between the lower layer of TPU and the upper layer of fluid impermeable material. The lower layer of TPU is adjacent and fixed to the primary material of the main body. The protection panel is sized and configured to prevent leakage of bodily fluid from the garment when worn by a wearer.

### CROSS REFERENCE TO RELATED APPLICATIONS

Not applicable

### FIELD OF THE INVENTION

The present invention relates to under garments, and more particularly to absorbent and leak proof garments that absorb bodily fluids such as from incontinence, menstruation, reproductive systems or other conditions.

### BACKGROUND OF THE INVENTION

Viscose material is widely used as a fabric for under garments. Viscose is inexpensive, light and breathable, and can be readily washed and reused many times. However, viscose is inherently porous and does not retain fluid. Thus, wearers who suffer from incontinence or other conditions that cause loss of bodily fluids must additionally wear absorbent pads or even diapers in order to retain leaked bodily fluids.

Efforts have been made to provide unitary or one-piece underwear that absorbs body fluids. Examples include U.S. Pat. No. 8,117,675 and U.S. Pat. No. 8,460,265. For example, U.S. Pat. No. 8,460,266 discusses a need for a washable female undergarment designed primarily to handle minimal leakage. The minimal leakage may occur just prior to or after menstruation, while sleeping, or during periods of high activity, such as exercising or playing a sport.

While efforts have been made to provide under garments that absorb body fluid, such garments may suffer from limited absorbency or leakage. Such garments can be bulky or otherwise uncomfortable to wear. Additionally, durability and reusability may be limited. For example, repeated washing renders the garment ineffective at absorbing and retaining bodily fluids. There is a need for an improved undergarment having the characteristics and properties described herein.

### OBJECTS AND SUMMARY OF THE INVENTION

It is an object of the inventions to provide incontinence under garments that provide improved leak proof and absorptive functionality.

It is another object of the inventions to provide incontinence under garments that are durable and can be worn repeatedly after washing.

It is yet another object of the inventions is to provide incontinence under garments that are comfortable to wear, reduce bulk and plastic waste, and can be worn under conventional clothing.

The foregoing and other objectives are achieved by providing incontinence under garments having the features described herein.

The invention comprises, generally, a garment for absorbance and leak retention of bodily fluids discharged from a body of a wearer. The garment has a main body, the main body having a trunk opening on an upper end rimmed with an elastic waist band and a pair of leg openings along opposing lower edges. Each of the leg openings is rimmed with an elastic leg band. The main body has a front side and a back side, and an inner surface and an outer surface. The main body comprises a primary material. The garment includes a protection panel lining the inner surface of the main body along a center of the main body. The protection panel has a front portion along the front side and a back portion along the back side of the main body. The protection panel is sized and configured to prevent leakage of bodily fluid from the garment when worn by a wearer. The protection panel comprises a lower layer of thermoplastic polyurethane (TPU), an upper layer of a fluid permeable material, and an absorbent layer sandwiched between the lower layer of TPU and the upper layer of fluid impermeable material. The lower layer of TPU is adjacent and fixed to the primary material of the main body.

In embodiments, the leg bands are rimmed with TPU along the protection panel to thereby serve as a barrier to prevent leakage of bodily fluid from inside of the under garment and help drive fluid into the absorbent layer. In embodiments, the TPU layer is heat bonded to the main body. The primary material of the main body can be viscose. In embodiments, the primary material of the main body comprises bamboo, cotton, spandex, hemp, polyester, or modal. In embodiments, the primary material of the main body comprises between about 1 to about 99 percent viscose, and can further comprise at least one of bamboo, cotton, spandex, hemp, polyester, or modal or a combination thereof

The garment can be configured as an under garment, such as female underwear. In embodiments, the front portion of the protection panel extends from about a mid-portion of the front to about a crotch area of the garment. In embodiments, an upper edge of the front portion is curved upward in order to provide additional protective area. In embodiments, the front portion of the protection panel extends to the top of the waist band. In embodiments, the back portion of the protection panel extends downward and rearward from the front portion, providing a continuous protection panel. In embodiments, the back portion of the protection panel extends to the waist band and is incorporated into the waist band. In embodiments, the back portion has a generally six-sided configuration, with an opposing pair of substantially straight edges extending from the respective leg bands to the waist band and the remaining outer surface of the back of the garment is the primary material.

The protection panel can cover about seventy percent of the main body. In embodiments, the TPU outlines an entire outer edge of the protection panel. In embodiments, in order to provide a desirable amount of elasticity to the leg openings, the TPU does not extend into the upper portions of the leg openings above the protection panel. In embodiments, in order to provide a desirable amount of elasticity to the waist band, the TPU in the waist band does not extend beyond the protection panel. The foregoing and other objects, features, aspects and advantages of the invention will become more apparent from the following detailed description of the invention when considered in conjunction with the accompanying drawings.

### BRIEF DESCRIPTION OF THE DRAWINGS

FIG. 1 is a front view of one preferred embodiment of an under garment of the invention.

FIG. 2 is a back view of one embodiment of an under garment of the invention.

FIG. 3 is a pattern view showing one embodiment of an under garment of the invention in a dissembled arrangement.

FIG. 4 is a side cross-section view taken along A-A of FIG. 1.

FIG. 5 is a side-cross section detail view of the area encircled in FIG. 4, showing details of layers of materials used in an embodiment of a protection panel of the invention.

FIG. 6A is a top view of a waist band comprising fluid impermeable material throughout the waist band.

FIG. 6B is a top view of a waist band comprising fluid impermeable material along a portion of the waist band and purely elastic material along a remaining portion of the waist band.

FIG. 7A is a top view of a leg band comprising fluid impermeable material throughout the leg band.

FIG. 7B is a top view of a leg band comprising fluid impermeable material along a portion of the leg band and purely elastic material along a remaining portion of the leg band.

### PREFERRED EMBODIMENTS OF THE INVENTION

In the following detailed description of the preferred embodiments, reference is made to the accompanying drawings which form a part hereof, and in which are shown by way of illustration specific embodiments in which the invention may be practiced. It is to be understood that other embodiments may be used and structural changes may be made without departing from the scope of the present invention.

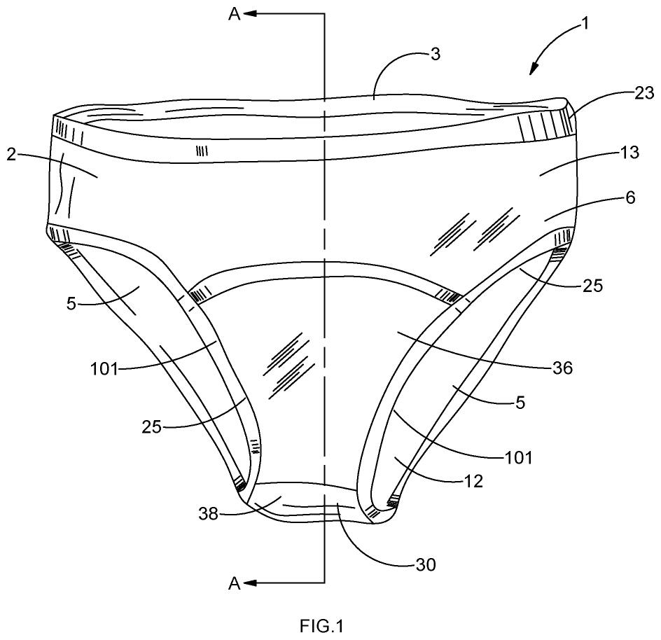

As shown in FIG. 1, the invention comprises generally an under garment 1 having certain configurations that are designed to prevent leakage of body fluids emitted from the body of a wearer, as described herein. As used herein, “bodily fluids” includes fluids emitted from the human body, including urine due to incontinence, blood from menstruation, reproductive system leakage, or fluids emitted due to other bodily conditions. In embodiments, the under garment 1 has the general configuration of an under garment, such as the pair of panties shown in FIG. 1. However, different configurations for the garment 1 can be used, as described below.

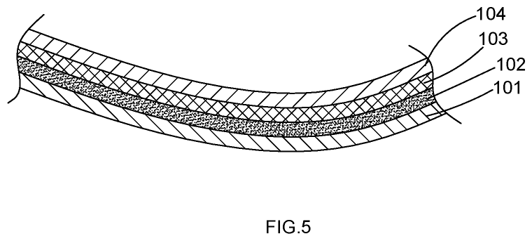

In the embodiment of FIG. 1, the main body 2 of the garment 1 has the configuration of a conventional pair of female underwear or panties. The main body 2 includes a trunk opening 3 and a pair of leg openings 5 arranged along opposing sides of the garment 1. The main body 2 includes a front side 6 and a back side 8, as well as an inner surface 12 and an outer surface 13. In embodiments, the entire main body 2 of the garment 1 is made of a primary material 101 (see FIG. 5). The primary material 101 can comprise viscose, bamboo, cotton, spandex, hemp, polyester, modal, or other fabric materials, or any variation of the listed materials in any amount or proportions. In embodiments, the primary material 101 can comprise anywhere from 1 percent to about 100 percent viscose, together with one or more of the materials listed above.

In addition to the above conventional features, the garment 1 has additional features that make it ideal for retaining bodily fluids. The garment 1 includes a large protection panel 30 extending generally along the center of the main body 2. The protection panel 30 includes layers of TPU 102, an absorbent material 103, and a viscose inner layer 104, as described in further detail below, particularly with reference to FIG. 5. In the embodiment of FIG. 1, the protection panel 30 includes a front portion 36 and a rear portion 38. The front portion 36 of the protection panel 30 extends downward from about a mid-portion of the front 6 to about the pubic or crotch area of the garment. In alternative embodiments, the front portion 36 can extend to the top of the front waist band. As shown in FIG. 1, an upper edge of the front portion 36 may be curved upward in order to provide additional protective area. A back portion 38 of the protection panel 30 extends downward and rearward from the front portion 36, providing a continuous protection panel extending along at least the regions shown in the drawings.

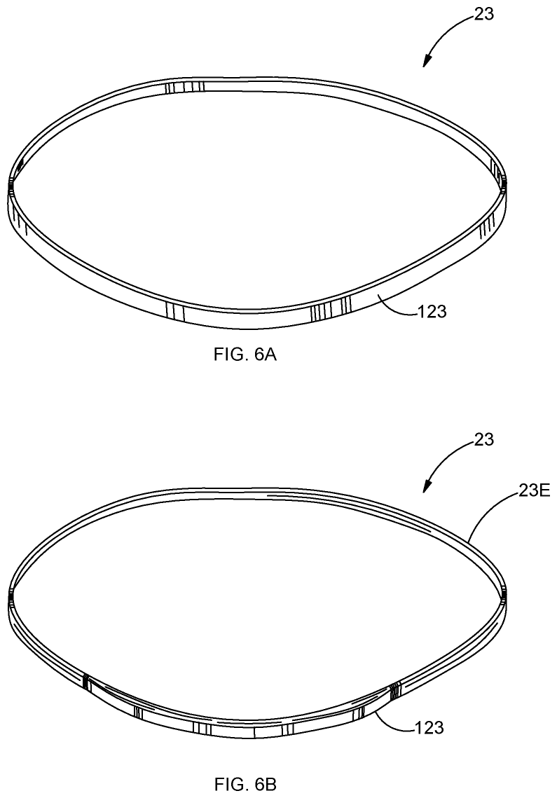

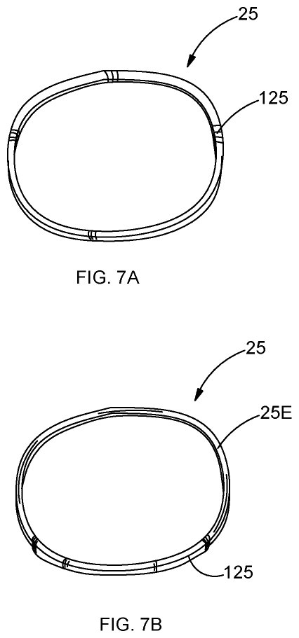

Additionally, and as discussed further below with reference to FIGS. 6A-7B, in embodiments the trunk opening 3 and leg openings 5 are rimmed with an elastic waist band 23 and elastic leg bands 25, in the conventional manner. However, the waist band 23 or the leg bands 25 are additionally lined with TPU in selected areas, such as along the protection panel 30. The TPU acts as a retainer or barrier to prevent leakage of bodily fluid from inside of the under garment 1. This helps drive fluid into the protection panel 30.

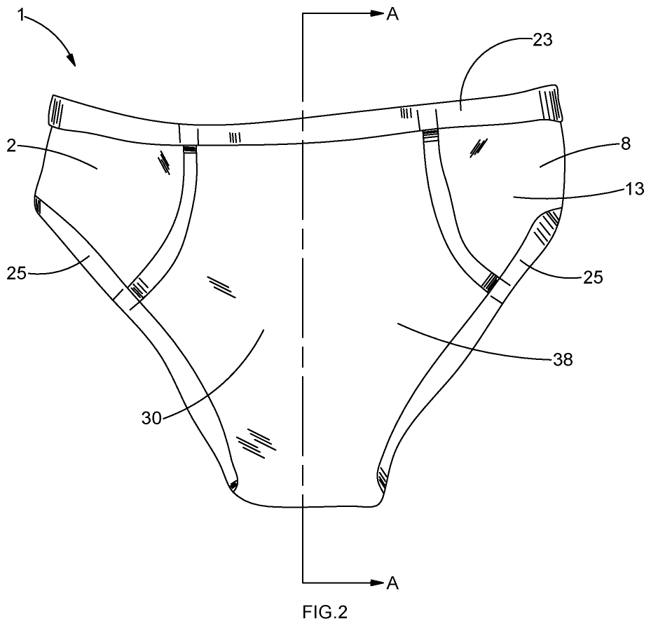

FIG. 2 is a back view of one embodiment of an under garment 1 of the invention, representing the back view of the garment 1 shown in FIG. 1. In the embodiment of FIG. 2, the back portion 38 of the protection panel 30 extends all the way to the top of the garment 1, where it can be incorporated into the waist band 23. In the embodiment of FIG. 2, the back portion 38 has a generally six-sided configuration, with an opposing pair of substantially straight edges extending from the respective leg bands 25 to the waist band 23. The remaining outer surface 13 of the back 8 of the garment 1 is formed by the viscose material 101, as described above. The back portions of the leg bands 25 include TPU material for retention of fluid, as described herein. In embodiments, the protection panel 30 covers about seventy percent of the main body 2.

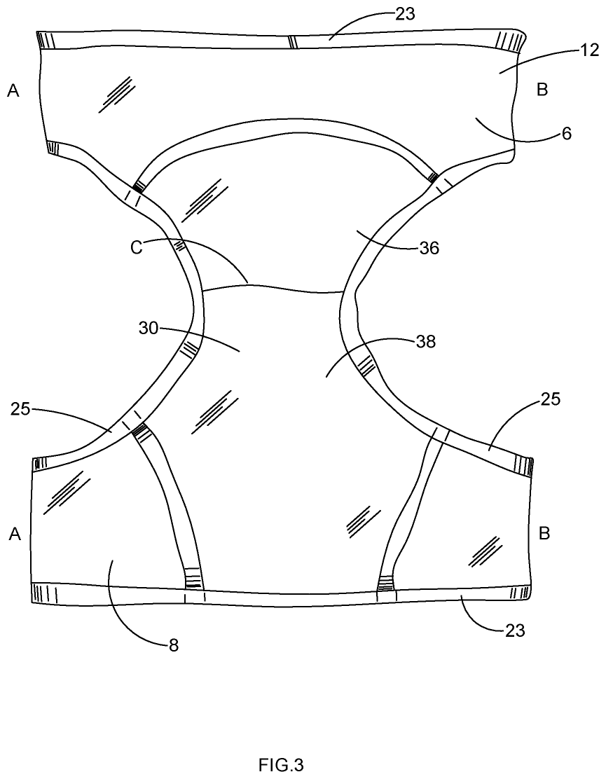

FIG. 3 is a pattern view showing one embodiment of an under garment 1 of the invention in a dissembled arrangement. In the embodiment of FIG. 3, the main body 2 of the garment 1 has an hour-glass configuration, which is a conventional configuration for underwear. It will be appreciated that when fully assembled, seam A is joined to opposing seam A, while seam B is joined to opposing seam B, thus forming the assembled embodiments shown in FIGS. 1-2. However, unlike conventional underwear, a selected portion of the inner surface 12 of the main body 2 is overlaid with a protection panel 30 that is sized and configured to prevent leakage of bodily fluids from the garment when worn by a wearer. In embodiments, a substantial portion or all of the protection panel 30 is layered in the manner discussed with respect to FIG. 5 below.

In the embodiment of FIG. 3, the panel includes a front portion 36 and a back portion 38. The front portion 36 is smaller than the back portion 38. The front portion 36 extends upward from seam C upward toward the waist band 23. However, as can be seen in FIGS. 1 and 3, the front portion 36 does not extend all the way to waist band 23, since a protection panel is typically not required in the front upper portion of the garment 1, although in embodiments, the front portion 36 can extend all the way to the waist band 23. As indicated in FIG. 3, the main body 2 can consist of an upper portion 6 joined to the lower portion 8 along seam C. In embodiments, the seam is only on the outside of the underwear, not the inside. The seam can also be eliminated, depending on the style of the garment 1.

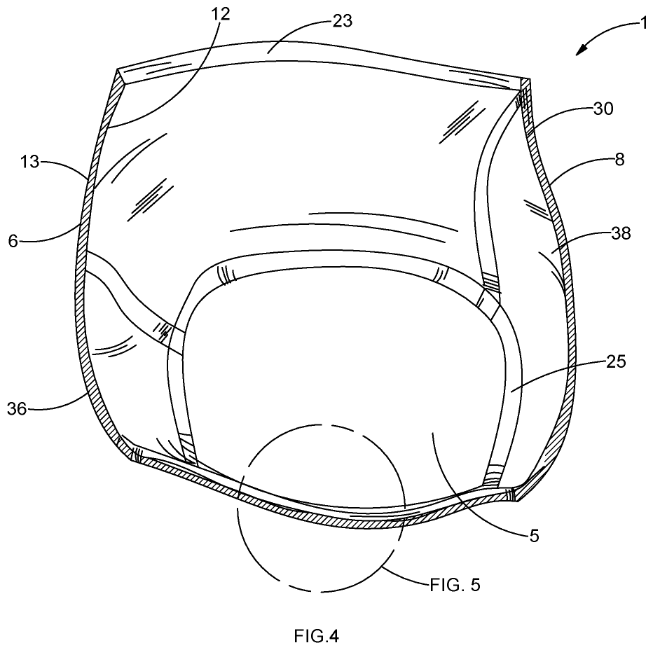

FIG. 4 is a side cross-section view taken along A-A of FIG. 1. In this view, the back portion 38 of the protection panel 30 can be seen extending all the way from the waist band 23, around the bottom of the garment, and connecting to the front portion 36, which extends approximately midway up the front 6 of the garment. As indicated in FIG. 4, a band of TPU is also provided in the leg bands 25 around the leg openings 5 of the garment, to further prevent fluid from leaking out of the protection panel 30. In embodiments, TPU outlines the entire outer edges of the protection panel, including along the top and opposing sides/leg bands 25 of the front portion 36, and the top side, upper opposing sides, and lower opposing sides/leg openings of the back portion 38. However, in order to provide a desirable amount of elasticity to the leg openings 5, in embodiments, the TPU does not extend into the upper portions of the leg openings above the protection panel 30; the upper portions of the leg bands 25 are provided with internal elastic bands in the conventional manner. Similarly, in embodiments, in the waist band 23, TPU is used only in the portion of waist band 23 that extends into the protection panel 30, while the remainder of the waist band 23 is provided with elasticity. The dotted line in FIG. 4 indicates a cut-out cross section of the protection panel 30, which is discussed in detail below with reference to FIG. 5.

In embodiments, the absorbent layer 103 extends into the waist band 23 and/or the leg bands 25. In embodiments, the absorbent layer 103 can overlay the TPU outline in the waist band 23 and/or the leg band 25.

FIG. 5 is a side-cross section view showing one embodiment of layers of materials used in the undergarments of the invention 1. FIG. 5 provides a closeup of the layers encircled in FIG. 4. A protection panel 30 is provided on an inner surface 12 of the undergarment 1. The protection panel 30 includes an outer layer 101, a fluid impermeable layer 102 (e.g. TPU or PUL) overlying and adjacent the outer layer 101, an absorbent layer 103 overlying and adjacent the fluid impermeable layer 102, and a fluid permeable inner layer 104 (e.g. viscose) overlying, adjacent to, and generally enclosing the absorbent material 103. In embodiments, the outer layer 101 comprises the fabric of the main body 2 and may be made of viscose or viscose in combination with other fabric materials, as described herein. In embodiments, the other three layers are overlaid on and attached to the outer layer 101. In embodiments, the absorbent layer 103 is adhered to the inner surface of the fluid permeable inner layer 104. In this manner, bodily fluid passes through the viscose inner layer 104 and is absorbed by the absorbent layer 103, where the fluid is captured until washing. The fluid impermeable layer 102 prevents the fluid from seeping from the absorbent layer 103 and through the outer layer 101, where the fluid would soil the outer layer and overlying garments, such as a pair of pants, shorts or a skirt. In embodiments, the fluid impermeable layer 102 is TPU and the TPU is glued to the outer layer.

The absorbent layer 103 does not include TPU. In embodiments, the absorbent layer 103 includes one, two, or a plurality of layers of towels. In embodiments, the towels are terry cloth or polyester-cotton blend, for ultra-absorbency. In embodiments, the towels are about 80 percent polyester and about 20 percent nylon. Other materials may be used for the absorbent layer 103 provided that the absorbent layer 103 absorbs bodily fluids and can be washed and reused. The TPU layer 102 is the bottom layer of the protection panel 30 overlaying the main body 2 of the underwear.

Additional details about embodiments of the waist 23 and leg bands 25 are provided in FIGS. 6-7. FIG. 6A is a top view of a waist band 23 comprising fluid impermeable material 123 such as TPU throughout the waist band. In the alternative embodiment of FIG. 6B, the waist band 23 comprises fluid impermeable material 123 along a portion of the waist band and elastic material 25E along a remaining portion of the waist band. In other embodiments, the fluid impermeable material 123 is adjacent to an elastic material or band 23E along all or part of the length of the fluid impermeable material 123. In this embodiment, the elastic material 23E is preferably adhered to the fluid impermeable material 123 such as by heat bonding, gluing or stitching. The fluid impermeable-elastic combination 123-23E is particularly advantageous when using TPU as the fluid impermeable material, because TPU is not elastic.

Similarly, FIG. 7A is a top view of a leg band 25 comprising fluid impermeable material 125 throughout the leg band. In the alternative embodiment of FIG. 7B, the leg band comprises fluid impermeable material 125 along a portion of the leg band and purely elastic material 25E along a remaining portion of the leg band. In other embodiments, the fluid impermeable material 125 is adjacent to an elastic material or band 25E along all or part of the length of the fluid impermeable material 125. In this embodiment, the elastic material 25E is preferably adhered to the fluid impermeable material 125 such as by heat bonding, gluing or stitching. The fluid impermeable-elastic combination 125-25E is particularly advantageous when using TPU as the fluid impermeable material, because TPU is not elastic.

As discussed herein, the TPU layer and the linings around the openings prevents bodily fluids from seeping out of the bottom or sides of the garment 1. Although not conventionally used in fabrics or garments, TPU is a waterproof/water impermeable material that is flexible and can be rewashed. Other fluid impermeable materials can be used as alternatives to TPU, such as polyurethane laminate (PUL). The alternative fluid impermeable materials such as PUL can be used for the fluid impermeable layer 102, the leg bands, the waist bands, or some or all of these components. TPU is heat bonded to the garment and is more flexible than PUL. PUL is chemically bonded to form a laminate fabric. TPU requires more care than PUL, such as when laundering the garment. However, TPU has the advantage of being less expensive than PUL. TPU is also bio-degradable and is free of chemical solvents.

The briefs of the invention 1 provide a breathable and comfortable one-piece protective undergarment that can be washed and reused many times. The garment 1 prevents or reduces leakage, seepage or bleed-through to outer clothing while also providing comfort to the wearer.

While the invention is described herein as an under garment that prevents leakage due to incontinence, the concepts of the invention can be applied to other garments. Examples include but are not limited to athletic shorts that incorporate the configuration described herein, sweatpants that incorporate a leak proof lining, skirts that incorporate a leak proof lining (e.g. tennis skirts), thongs, boy shorts, athletic briefs, pants, underwear, swim suits, sleep wear, and the like.

In operation, the garment 1 of the invention is worn like a conventional pair of garments. However, if bodily fluid discharges from the wearer, the bodily fluid flows through the fluid permeable layer and into the absorbent layer, where it is substantially retained by the fluid impermeable layer or the fluid impermeable leg or arm bands. The configuration of the garment 1 keeps the wearer dry and prevents bodily fluids from soiling the wearer's outer clothing. When the wearer changes clothes, the wearer removes the garment 1 and washes it in a conventional manner. The garment 1 can be washed and reused many times, in the nature of conventional garments but while retaining the ability to absorb and prevent leakage of bodily fluids from the garment 1.

Although the present invention has been described in terms of specific embodiments, it is anticipated that alterations and modifications thereof will no doubt become apparent to those skilled in the art. It is therefore intended that the following claims be interpreted as covering all alterations and modifications that fall within the true spirit and scope of the invention.

What is claimed is:

1. A garment for absorbance and leak retention of bodily fluids discharged from a body of a wearer, comprising:
a main body, the main body having a trunk opening on an upper end rimmed with an elastic waist band, a pair of leg openings along opposing lower edges, each of the leg openings rimmed with an elastic leg band, a front side and a back side, an inner surface and an outer surface, the main body comprising a primary material,
a protection panel lining the inner surface of the main body along a center of the main body, the protection panel having a front portion along the front side and a back portion along the back side of the main body, the protection panel sized and configured to prevent leakage of bodily fluid from the garment when worn by a wearer,
the protection panel comprising a lower layer of thermoplastic polyurethane (TPU), an upper layer of a fluid permeable material, and an absorbent layer sandwiched between the lower layer of TPU and the upper layer of fluid impermeable material, the lower layer of TPU adjacent and fixed to the primary material of the main body.

2. The garment of claim 1, further comprising the leg bands rimmed with TPU along the protection panel to thereby serve as a barrier to prevent leakage of bodily fluid from inside of the under garment and help drive fluid into the absorbent layer.

3. The garment of claim 1, wherein the TPU layer is heat bonded to the main body.

4. The garment of claim 1, wherein the primary material of the main body is viscose.

5. The garment of claim 1, wherein the primary material of the main body comprises bamboo, cotton, spandex, hemp, polyester, or modal.

6. The garment of claim 1, wherein the primary material of the main body comprises between about 1 to about 99 percent viscose.

7. The garment of claim 6, wherein the primary material of the main body further comprises at least one of bamboo, cotton, spandex, hemp, polyester, or modal or a combination thereof.

8. The garment of claim 1, wherein the garment is configured as an under garment.

9. The garment of claim 8, wherein the under garment is female underwear.

10. The garment of claim 1, wherein the front portion of the protection panel extends from about a mid-portion of the front to about a crotch area of the garment.

11. The garment of claim 10, wherein an upper edge of the front portion is curved upward in order to provide additional protective area.

12. The garment of claim 1, wherein the front portion of the protection panel extends to the top of the waist band.

13. The garment of claim 1, wherein the back portion of the protection panel extends downward and rearward from the front portion, providing a continuous protection panel.

14. The garment of claim 13, wherein the back portion of the protection panel extends to the waist band and is incorporated into the waist band.

15. The garment of claim 1, wherein the back portion has a generally six-sided configuration, with an opposing pair of substantially straight edges extending from the respective leg bands to the waist band and the remaining outer surface of the back of the garment is the primary material.

16. The garment of claim 1, wherein the protection panel covers about seventy percent of the main body.

17. The garment of claim 1, wherein the TPU outlines an entire outer edge of the protection panel.

18. The garment of claim 1, wherein, in order to provide a desirable amount of elasticity to the leg openings, the TPU does not extend into the upper portions of the leg openings above the protection panel.

19. The garment of claim 1, wherein, in order to provide a desirable amount of elasticity to the waist band, the TPU in the waist band does not extend beyond the protection panel.
